(19) United States
(12) Patent Application Publication
(10) Pub. No.: US 2003/0026761 A1
(43) Pub. Date: Feb. 6, 2003

(54) Method for evaluating or diagnosing skin condition based on intercellular structure

(75) Inventors: Daniel Thorn Leeson, Hoboken, NJ (US); Krishna Kumar Subramanyan, Edgewater, NJ (US); Catherine Lynn Meyers, Glen Rock, NJ (US)

Correspondence Address: UNILEVER, PATENT DEPARTMENT, 45 RIVER ROAD, EDGEWATER, NJ, 07020, US

(73) Assignee: Unilever Home & Personal Care, Division of Conopco , Inc.

(21) Appl. No.: 09/920,794

(22) Filed: Aug. 2, 2001

(51) Int. Cl. A61K 49/00

(52) U.S. Cl. 424/9.6

(57) ABSTRACT

This invention relates to a method for evaluating or diagnosing the condition of skin based on the intercellular structure of skin as well as the intercellular distribution of particular cellular properties of skin.

### FIELD OF THE INVENTION

The present invention relates to methods for evaluating or diagnosing skin condition by evaluating intercellular structure or distribution and then comparing to skin condition which has been determined by a different methodology.

### BACKGROUND OF THE INVENTION

There are numerous methods available for evaluating the condition of human skin. Although a few methods are available that are based on specific intracellular features of skin cells (see, for instance, Skin optics revisited by in vivo confocal microscopy: Melanin and sun exposure P. Corcuff et al. J. Cosmet Sci. 52 (2001) 91, where changes in basal keratinocytes are observed after sun exposure), none of these methods consider the intercellular structure of the stratum corneum, i.e. the way the cells are arranged spatially with respect to each other, or consider the intercellular distributions of specific cellular properties (e.g., absorbance of dye on skin cells). On the other hand, methods that do take into account the specific structure or organization of the skin surface or stratum corneum as a whole (see, for instance, Skin surface patterns of xerotic legs: the flexural and accretive types C. Piérard-Franchimont et al. Int J. Cosmet. Sci. 23 (2001) 121, where specific patterns of alteration of the skin surface are assigned to different types of leg dryness), do not consider the structure of the skin surface at the cellular level.

### BRIEF DESCRIPTION OF THE INVENTION

The present invention relates to a method to evaluate skin condition based on intercellular organization of the cells.

The present invention discloses one specific embodiment for an evaluation of skin condition.

In the embodiment of the present invention the intercellular structure of the skin surface is evaluated by choosing a desired spot on a subject's body; applying a contrast-enhancing fluorescent dye to the chosen spot; measuring the intercellular structure of the chosen area (e.g., by taking an image with a scanning confocal fluorescence microscope); evaluating the intercellular structure of the skin surface either by visual inspection or mathematical analysis of the resulting image; and, comparing the intercellular condition of the skin surface to skin condition which has been determined by a different methodology (e.g., a clinical evaluation of skin dryness by a certified clinical grader).

More specifically, the invention comprises a method to evaluate or diagnose skin condition based on intercellular structure and the intercellular distribution of cellular properties. The method comprises:

1. choosing a desired site, typically 1×1″ in size, although it should be understood the site can be either larger or smaller, on the body of a subject;

2. measuring the intercellular organization of skin cells or intercellular distribution of particular cellular properties at the chosen site by any appropriate method (e.g., using contrast enhancing fluorescent dye and acquiring image; such method is disclosed for example in applicants co-pending application, filed same date entitled “Method for Imaging Skin Surface Intercellular and Intracellular Structure Using a Compound to Enhance Contrast”);

3. evaluating the intercellular organization of the skin surface at the chosen site according to a protocol determined by the specific method used in step (2);

4. comparing the evaluation of intercellular organization done in step (3) to a known specific skin condition measured by different methodology.

### BRIEF DESCRIPTION OF THE DRAWINGS

FIG. 1a displays two-dimensional top views of the intercellular structure at lower outer leg of four different subjects with varying degrees of natural dryness.

FIG. 1b is a schematic drawing of both top view and side view of dry (top and bottom left) and healthy (top and bottom right) skin as they undergo desquamation process.

FIG. 2 displays two two-dimensional-top views of the intercellular structure at the lower outer leg of the same subject. The image on the left was taken immediately before a 5-day treatment phase with a leave-on moisturizer, the image on the right was taken immediately after.

FIG. 3 displays three two-dimensional top views of the intercellular structure at the upper volar forearm of the same subject at different points during a 5-day treatment phase with a harsh soap bar. The image on the left was taken on the first day, prior to any treatment. The middle image was taken on the third day. The image on the right was taken on the fifth day, after the final treatment.

### DETAILED DESCRIPTION OF THE INVENTION

The present invention relates to a method for evaluating or diagnosing skin condition, based on intercellular structure and the intercellular distribution of cellular properties.

Specifically, the invention provides a method for evaluating or diagnosing skin condition, based on intercellular structure and the intercellular distribution of cellular properties, comprising:

1. choosing a desired site, typically 1×1″ in size but the site can be either larger or smaller, on the body of a subject;

2. measuring the intercellular organization or intercellular distribution of specific cellular properties of the skin at the chosen site by any appropriate method, in vivo or ex vivo, for example by applying a contrast enhancing fluorescence dye and taking images with a scanning confocal fluorescence microscope;

3. evaluating the intercellular organization or intercellular distribution of specific cellular properties of the skin at the chosen site according to a protocol determined by the specific method used in step (2);

4. comparing the evaluation of intercellular organization done in step (3) to a known skin condition, for example skin dryness, although it should be understood this can be any measure of skin condition including medical conditions.

Each of the process steps is discussed in more detail below and in the examples.

As noted, the first step in the method for evaluating skin condition based on the intercellular structure and on the intercellular distribution of cellular properties, is to choose a desired spot on the body of the subject.

In principal, there are no restrictions on the size or the shape of the body site that is chosen. Certain restrictions may be imposed by the specific measurement technique used in the subsequent step. Practical considerations in choosing the size of the body site may be the expected variations in intercellular properties between body sites. Generally speaking, it may be advisable to chose a size of the body site approximately on the length scale of the variations that may be expected.

In the second step of the invention, the intercellular features of the body site that was chosen in the first step are measured using an appropriate instrument.

The intercellular structure of skin essentially refers to the three-dimensional spatial arrangement of skin cells. For instance, the upper layers of the stratum corneum may comprise of well-defined layers of cells, with each layer forming a highly ordered lattice of regularly shaped, flatly lying cells. On the other extreme, the surface layers may comprise a highly disordered array of cells.

Ideally, the instrument will provide the complete three-dimensional arrangement of skin cells in all different layers. At present, no such instrument is available and the three-dimensional arrangement may have to be inferred from an alternative measurement, while specific instruments may have to be used to study different layers. In the embodiment of this invention a two-dimensional top view of the intercellular morphology in the upper layers of the stratum corneum is obtained by applying a contrast-enhancing fluorescent dye (examples include fluorescein, pyranine and fluorescein octadecyl ester) and subsequently taking fluorescence images with a scanning confocal fluorescence microscope. The resulting images are condensed into a single two-dimensional top view of the skin surface, that clearly reveals the outlines of the individual cells at the skin surface. This technique is described in applicants' co-pending application, entitled “Method for Imaging Skin Surface Intercellular and Intracellular Structure Using a Compound to Enhance Contrast”, filed on same date and hereby incorporated as reference into the subject application.

The intercellular distribution of cellular properties may also be an indicator of certain skin conditions. Examples of cellular properties are shape and size of the cells.

In the third step of the invention the intercellular structure or intercellular distribution of specific cellular properties of the skin at the chosen site are evaluated according to a protocol determined by the specific method used in the second step.

The evaluation of the intercellular structure of skin or intercellular distribution of specific cellular properties will be dependent on the method that is used in the second step. In the embodiment of this invention evaluation can be done by either visual inspection or mathematical analysis of two-dimensional top views of the intercellular arrangement.

In the fourth step of the invention, the evaluation of intercellular organization done in the third step is compared to a known skin condition which has been determined or diagnosed by a different methodology. An example of this includes skin dryness, although can be any measure of skin condition including medical conditions.

In the embodiment of this invention, the intercellular organization at the skin surface is compared to and shown to correlate with skin dryness. Alternatively, the intercellular organization of the skin surface and stratum corneum may, in part, determine visual and tactile properties of skin or may prove to be an indicator of certain medical skin conditions, such as psoriasis. As such, the intercellular organization of the upper layers of the stratum corneum may be used as a diagnostic measure of different skin conditions.

Methodology

Equipment

Stacks of fluorescence as well as reflectance images were taken with a Noran In Vivo OZ scanning confocal microscope. Maximum intensity renderings of the stacks of images were performed with the image analysis software supplied with the instrument.

Experimental Procedure

A contrast enhancing fluorescent dye, fluorescein, was applied topically by soaking a circular area of the skin, 0.5″ in diameter, for two minutes with 200 μl of an aqueous solution at a concentration of 1,000 ppm and a pH of 7.0. The skin was subsequently rinsed with tap water for 10 seconds and patted dry. Images were subsequently acquired with the in vivo confocal microscope.

Except in the operating and comparative examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

The following examples are intended to further illustrate the invention and are not intended to limit the invention in any way.

Unless indicated otherwise, all percentages are intended to be percentages by weight.

### EXAMPLE 1

Intercellular organization of the skin surface compared to natural variations in leg dryness

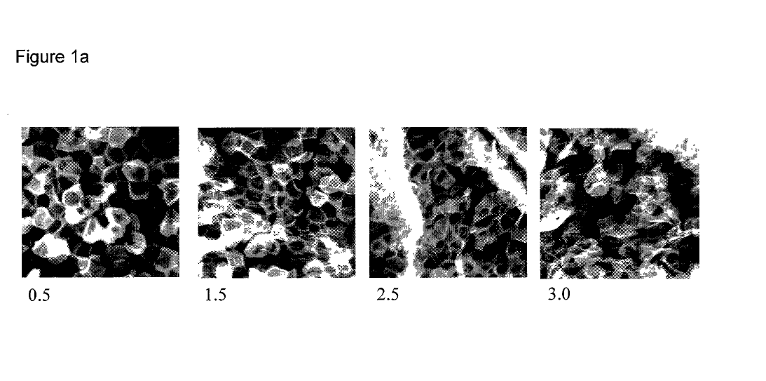

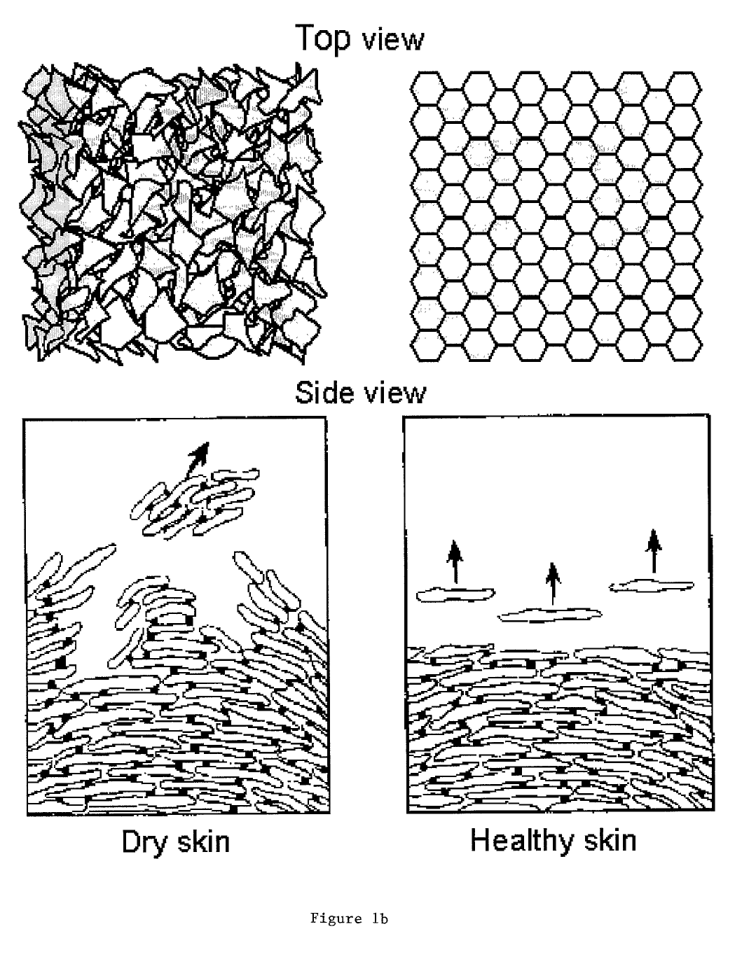

FIG. 1 shows two-dimensional top views, approximately 0.01×0.01″ in size, of the skin surface of the outer lower leg of four different female subjects. It is clear that the image on the far left and the image on the far right show two extreme cases of intercellular organization. The image on the far left shows a highly ordered arrangement of regularly shaped surface cells while the image on the far right shows a highly disordered pattern where it is hard to even distinguish individual cells. The other two images lie between these two extremes. Also shown in FIG. 1 are the visual dryness scores evaluated by a certified clinical grader. It is evident that the degree of order of the cellular patterns observed in the microscopic images correlates very well with the clinical dryness grade, with 0.5 being least dry and 3.0 being driest on a scale of 0-4 used by a trained visual evaluator.

It should be noted that, since dry skin is a result of dysfunctional desquamation of skin cells, it makes sense for the intercellular structure and the clinical dryness grade to be correlated. This is illustrated in FIG. 1b. For desquamation of skin cells to occur the desmosomal bonds between them need to be digested by enzymes. In healthy skin, these enzymes are active and cells are shedded individually, which causes the ordered layer structure of skin cell to remain intact (top right and bottom right figures in FIG. 1b). Consequently, a top view of healthy skin will appear as a highly ordered array of flat-lying cells, similar to the image on the far left in FIG. 1a. In dry skin, because of inhibited activity of the desmosome-digesting enzymes, cells accumulate at the surface as clumps leading to a highly disordered arrangement of cells (top left and bottom left figures in 1b). The top view of such a disordered arrangement is expected to appear as the image on the far right in FIG. 1a.

### EXAMPLE 2

Changes in intracellular organization of the skin surface induced by treatment with a leave-on moisturizer

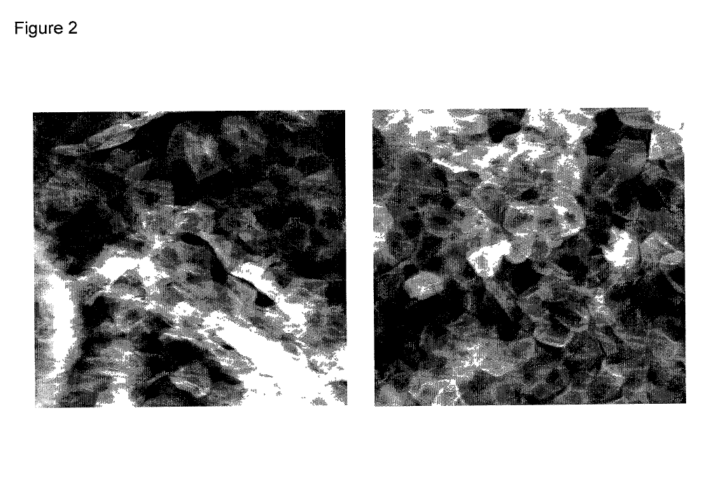

FIG. 2 shows two two-dimensional top views of the skin surface at the same body site, the lower outer leg, of the same subject. The image on the left was taken before a 5-day treatment phase where a glycerol/water moisturizer was applied twice-daily. The image on the right was taken immediately after the treatment phase. It appears that the moisturizer treatment induces a significant improvement in the intercellular organization based on the specific features discussed in Example 1.

### EXAMPLE 3

Changes in intracellular organization of the skin surface induced by treatment with a harsh soap bar

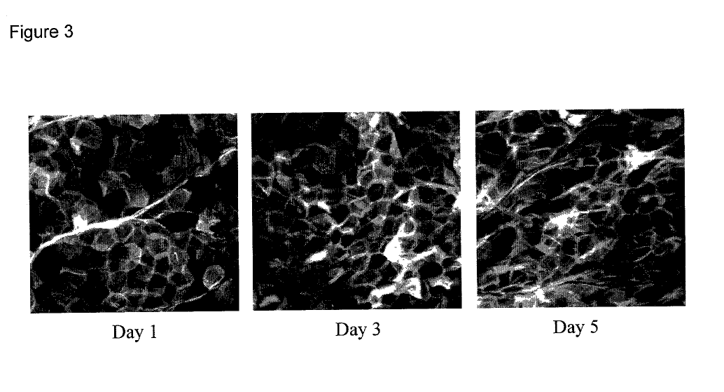

FIG. 3 shows three two-dimensional top views of the skin surface at the same body site, the upper volar forearm, of the same subject, at different points during a 5-day treatment phase, where the site was washed for two minutes, four times a day, with a harsh soap bar, known to cause dryness after repeated usage. The image on the left was taken on the first day, prior to any treatment. The middle image was taken on the third day. The image on the right was taken on the fifth day, after the final wash. It is evident that the treatment induces significant changes, predominantly an increasing degree of disorder of the intercellular pattern, based on the specific features discussed in Example 1.

1. A method for evaluating or diagnosing the condition of skin based on the intercellular structure of skin as well as the intercellular distribution of particular cellular properties of skin, comprising:
(1) choosing a desired spot of any size or shape on the body of a subject;
(2) measuring the intercellular structure or intercellular distribution of particular cellular properties of the skin at the chosen site by any appropriate method;
(3) evaluating the intercellular structure or intercellular distribution of specific cellular properties of the skin at the chosen site according to a protocol determined by the specific method used in step (2);
(4) comparing the evaluation of intercellular organization or intercellular distribution of specific cellular properties done in step (3) to a skin condition which has been determined by a different methodology in order to evaluate or diagnose condition.

2. A method according to claim 1, wherein method of measuring of sub-paragraph (a) comprises applying contrast enhancing fluorescent dye, and acquiring changes.

3. A method according to claim 2, wherein dye is fluorescein.

4. A method according to claim 2, wherein image is acquired with confocal fluorescence microscope.

5. A method according to claim 1, wherein skin condition is skin dryness.
